(19) United States
(12) Patent Application Publication
(10) Pub. No.: US 2024/0366945 A1
(43) Pub. Date: Nov. 7, 2024

(54) ADAPTIVE REAL-TIME STATE SPACE CONTROL OF A NEUROSTIMULATION DEVICE

(71) Applicant: REGENTS OF THE UNIVERSITY OF MINNESOTA, Minneapolis, MN (US)

(72) Inventors: Theoden I. Netoff, Minneapolis, MN (US); Andrew Lamperski, Minneapolis, MN (US); Zachary Sanger, Minneapolis, MN (US); Alik Widge, Minneapolis, MN (US); David Darrow, Minneapolis, MN (US)

(21) Appl. No.: 18/635,934

(22) Filed: Apr. 15, 2024

Related U.S. Application Data
(60) Provisional application No. 63/459,344, filed on Apr. 14, 2023.

(51) Int. Cl. A61N 1/36

(52) U.S. Cl. CPC A61N 1/36139

(57) ABSTRACT

Neurostimulation, such as electrical and/or magnetic neurostimulation, is controlled using an adaptive real-time state space (ARTISTS) control framework to determine and/or adjust stimulation settings (e.g., stimulation waveforms). The ARTISTS control framework generally includes an adaptive autoregressive model of the nervous system's response to stimulation, an amended Kalman filter to estimate the state and coefficients of the autoregressive model, and a linear quadratic regulator to determine the stimulation waveform to be delivered.

### CROSS-REFERENCE TO RELATED APPLICATIONS

This application claims the benefit of U.S. Provisional Patent Application Ser. No. 63/459,344, filed on Apr. 14, 2023, and entitled “ADAPTIVE REAL-TIME STATE SPACE CONTROL OF A NEUROSTIMULATION DEVICE,” which is herein incorporated by reference in its entirety.

### BACKGROUND

Neurostimulation modulates the neural activity of a subject's nervous system, such as by applying electrical and/or magnetic fields to one or more anatomical targets in the subject's nervous system. The application of neurostimulation can be used to provide motor cortex stimulation, cranial nerve stimulation (e.g., vagus nerve stimulation), spinal cord stimulation, as well as treatment for neurological disorders, psychiatric disorders, and the like.

### SUMMARY OF THE DISCLOSURE

It is an aspect of the present disclosure to provide a method for determining stimulation settings for a neurostimulation device. The method includes measuring neural activity in a subject using a recording electrode, where the neural activity is created in response to a neurostimulation delivered to a neural target in the subject by a neurostimulation device. An autoregressive model is constructed based on the measured neural activity. A neural state of the subject is estimated based on the autoregressive model, where the neural state includes a real-time state of the subject's nervous system. Stimulation settings are determined based on the estimated neural state. Neurostimulation is then delivered to the neural target in the subject with the neurostimulation device using the stimulation settings. Other embodiments of this aspect include corresponding systems (e.g., computer systems, controllers, processors), programs, algorithms, and/or modules, each configured to perform the steps of the methods.

It is another aspect of the present disclosure to provide a controller for controlling a neurostimulation device. The controller includes an input that receives neural activity signals measured from a subject, a processor, and an output that receives stimulation settings from the processor and communicates the stimulation settings to a neurostimulation device. The processor receives the neural activity signals from the input; constructs an autoregressive model based on the neural activity signals; extracts coefficients from the autoregressive model; estimates a neural state based on the extracted autoregressive model coefficients; and determines stimulation settings based on the estimated neural state.

### BRIEF DESCRIPTION OF THE DRAWINGS

FIG. 1 is a flowchart of an example method for controlling the delivery of neurostimulation to a neural target in a subject.

FIG. 2 is a flowchart of an example adaptive real-time state space (ARTISTS) control framework for controlling a neurostimulation device.

FIGS. 3A-3C illustrate an example of using an ARTISTS control framework to minimize a signal.

FIG. 4 illustrates an example of using an ARTISTS control framework to minimize a signal in real time.

FIG. 5 is a block diagram of an example controller that can implement the systems and methods described in the present disclosure.

FIG. 6 is a block diagram of an example system for generating updated neurostimulation settings using an ARTISTS control framework.

FIG. 7 is a block diagram of example components that can implement the system of FIG. 6.

### DETAILED DESCRIPTION

Described here are systems and methods for controlling neuromodulation, such as electrical and/or magnetic neurostimulation. The neuromodulation can be provided for the treatment of neurological disorders, or other suitable applications of neuromodulation. In general, the systems and methods implement an adaptive real-time state space (ARTISTS) control framework to determine and/or adjust stimulation settings (e.g., stimulation waveforms) for use in controlling the operation of a neuromodulation and/or neurostimulation device. The ARTISTS control framework described in the present disclosure generally includes an adaptive autoregressive model of the nervous system's response to stimulation, an amended Kalman filter to estimate the state and coefficients of the autoregressive model, and a linear quadratic regulator to determine the stimulation waveform to be delivered.

It is an advantage of the ARTISTS control framework disclosed in the present disclosure to determine optimal neuromodulation settings to suppress pathological neural activity based on the state of the nervous system as determined by an adaptive model. Applications of this ARTISTS control framework (or a controller implementing the ARTISTS control framework) include, but are not limited to, electrical stimulation of neural targets for seizure suppression, Parkinson's disease, obsessive compulsive disorder, and essential tremor. In these cases, electrical stimulation may be used to suppress pathological neural activity.

Currently, neuromodulation is done with high amplitude pulses that are often delivered at fixed intervals independent of the state of the nervous system. Growing evidence has shown that stimulation based on the state of the nervous system, such as delivering stimulation when high amplitude activity is detected, as is done by the Responsive Neural Stimulation by NeuroPace, or delivered at particular phases of neural oscillations with millisecond precision, are as effective with less delivered energy, or more effective than open loop stimulation. It has also been shown that continuous closed-loop control with small amplitude perturbations in rapid response to current state estimation can be more effective at neuromodulation than pulsatile stimulation while using a fraction of the total energy delivered.

Continuous optimal stimulation waveforms can be determined given the state by using a model of the nervous system's response to stimulation. However, system identification of the nervous system's response to stimulation is often done independently of the controller. Accuracy and robustness of the controller will depend on how much the nervous system's dynamics change from the time of system identification and application of the controller, as well as changes in response to the application of the controller. The ARTISTS control framework described in the present disclosure is capable of continuously determining the model of the nervous system's response to stimulation through an adaptive model and regularly updates the controller to address non-stationarities of the nervous system. Furthermore, it does not depend on prior knowledge of the nervous system's response and builds the model as stimulation is applied.

As an example, an ARTISTS control framework is used to determine optimal stimulation settings to suppress pathological neural activity based on the state of the nervous system as determined by an adaptive model. In general, the ARTISTS control frame work uses an adaptive autoregressive model of the nervous system's response to stimulation, an amended Kalman filter to estimate the neural state, and a linear quadratic regulator to determine the stimulation waveform to be delivered based on the current estimate of the neural state.

Determining the optimal stimulation of the neural activity uses knowledge of the state of the nervous system as well as an accurate model to predict how the nervous system will respond to the stimulation. Neural activity can be measured from electrodes implanted in the brain and signals are digitized and stored on a device. The nervous system is also stimulated through the same electrodes, or different electrodes, by a neural stimulator. The ARTISTS control framework uses the response of the nervous system to applied electrical stimulation to adaptively fit an autoregressive model to minimize prediction error of the response to electrical stimulation. From this model, the controller then estimates the state of the nervous system at every sample. The model is also used to solve a linear quadratic regulator (LQR) algorithm that determines the optimal stimulation to apply from the current estimate of the neural state. This stimulus is then applied, using a neuromodulatory stimulation device to the nervous system through an electrode.

Advantageously, the ARTISTS control framework can be implemented on an implantable neural stimulator. In these instances, the ARTISTS control framework can provide closed-loop adaptive feedback to the nervous system through a neurostimulator. This controller can thus be translated to many neuromodulation treatment options where an implanted stimulator is the current standard of care.

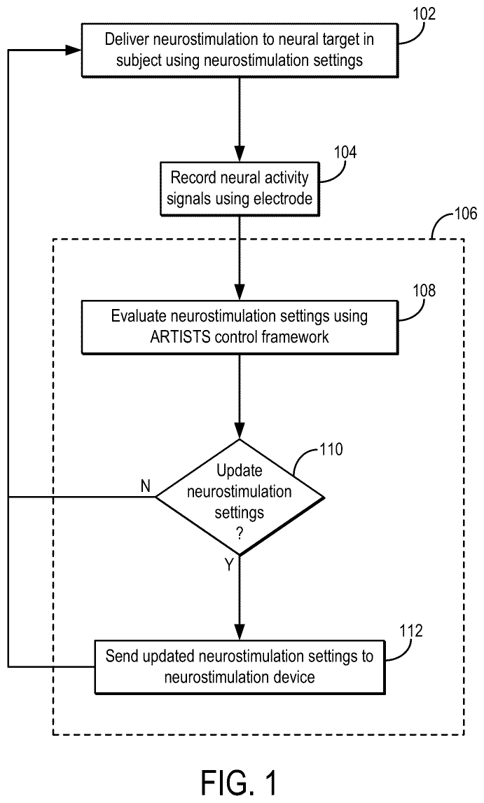

Referring now to FIG. 1, a flowchart is illustrated as setting forth the steps of an example method for controlling the delivery of neuromodulation (e.g., neurostimulation) to a neural target in a subject using an ARTISTS control framework.

The method includes delivering neurostimulation to a neural target in a subject using a set of neurostimulation settings, as indicated at step 102. For instance, the neurostimulation can be delivered using a neurostimulation device that generates the neurostimulation based on neurostimulation settings stored on the neurostimulation device. The neurostimulation settings can be initialized by a user, a clinician, a technician, or the like. Additionally or alternatively, the neurostimulation settings may be prestored initial neurostimulation settings, which may be tailored for the subject and/or the subject's neuropathology, or may be general neurostimulation settings.

While neurostimulation is being continuously delivered to a subject, neural activity signals are recorded with an electrode and stored as neural activity signal data, as indicated at step 104. The electrode may be a part of the neurostimulation device used to deliver neurostimulation to the subject, or may be a separate electrode. In general, the neural activity signal data are recorded while neurostimulation is being delivered to a neural target in the subject, or recorded in response to such neurostimulation. The neural activity signal data may include, for example, neural recordings of local field potentials in the neural target.

A controller of the neurostimulation device then analyzes the neural signal activity data using the disclosed ARTISTS control framework, as generally indicated at process block 106. This process includes evaluating at each sample of the neural activity signal data whether or not the neurostimulation settings should be updated based on the neural state of the subject to drive the neural activity to a target state, as indicated at step 108. When the controller determines that current neurostimulation settings should updated based on the evaluation of the neural state using the ARTISTS control framework, as generally indicated by decision block 108, then the updated neurostimulation settings generated using an ARTISTS control framework are sent to the neurostimulation device, as indicated at step 110. The updated neurostimulation settings are then used to deliver updated neurostimulation to the subject at step 102. For example, the neurostimulation settings may include updated neurostimulation waveforms that are sent to the neurostimulation device as a continuous voltage signal. If the evaluation of the neural state by the ARTISTS control framework determines that the neurostimulation settings do not need to be updated based on the most recent measurement of the neural activity signals in the subject, then the previous neurostimulation settings are continued to be used to deliver neurostimulation at step 102.

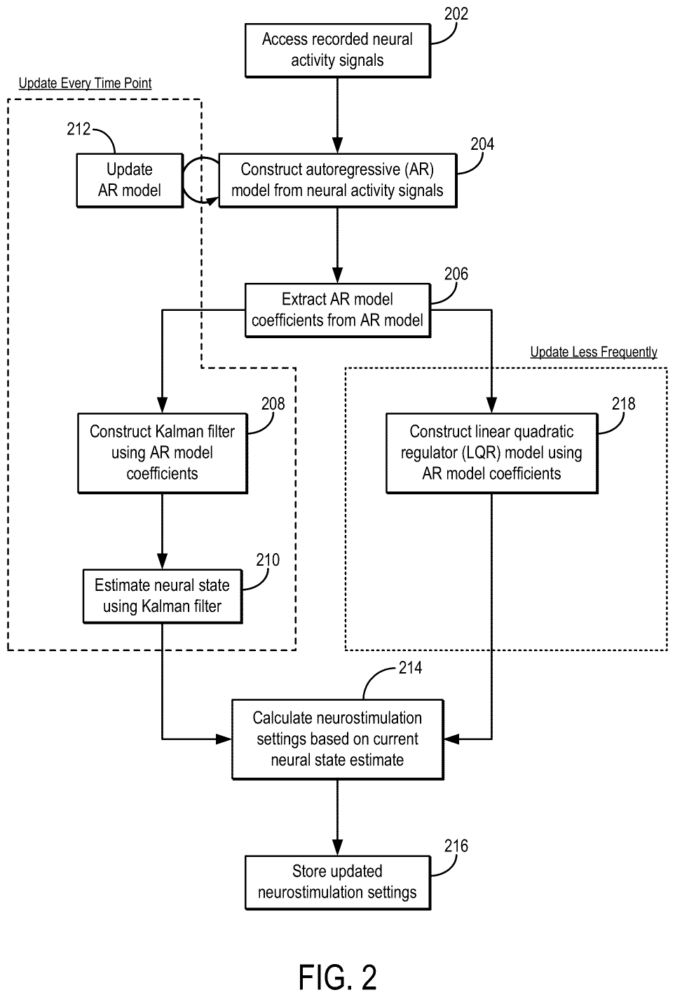

Referring now to FIG. 2, a flowchart is illustrated as setting forth the steps of an example method for updating neurostimulation settings using an ARTISTS control framework. The method includes accessing neural activity signal data recorded from a subject, as indicated at step 202. For example, the neural activity signal data can include neural activity signals recorded using an electrode. The electrode may be a part of the neurostimulation device used to deliver neurostimulation to the subject, or may be a separate electrode. In general, the neural activity signal data are recorded while neurostimulation is being delivered to a neural target in the subject, or recorded in response to such neurostimulation. The neural activity signal data may include, for example, neural recordings of local field potentials in the neural target.

An autoregressive (AR) model is then constructed from the neural activity signal data, as indicated at step 204. As a non-limiting example, an autoregressive predictor can be built with a time step, dₜ, formatted as:

$${d}_{t} = [ \begin{matrix}{u}_{t} \\ {z}_{t - 1} \\ ⋮ \\ {z}_{t - p}\end{matrix} ] to the output {y}_{t} , where {z}_{t} = [ \begin{matrix}{u}_{t} \\ {y}_{t}\end{matrix} ] . \qquad ( 1 )$$

The following objective function may be solved using recursive least squares:

$${min}_{G} {\sum }_{t = 1}^{T} {‖ {y}_{t} - {Gd}_{t} ‖}_{2}^{2} + {‖ G ‖}_{F}^{2} . \qquad ( 2 )$$

In particular when G₀=0, the following AR model coefficients can be determined to construct the AR model:

$${Ω}_{t + 1} = 1 + {d}_{t + 1}^{T} {P}_{t} {d}_{t + 1} ; \qquad ( 3 )$$

$${K}_{t + 1} = \frac{1}{{Ω}_{t + 1}} {d}_{t + 1}^{T} {P}_{t} ; \qquad ( 4 )$$

$${G}_{t + 1} = {G}_{t} + ( {y}_{t + 1} - {G}_{t} {d}_{t + 1} ) {K}_{t + 1} ; \qquad ( 5 )$$

$${P}_{t + 1} = {P}_{t} - {Ω}_{t + 1} {K}_{t + 1}^{T} {K}_{t + 1} . \qquad ( 6 )$$

AR model coefficients are then extracted from the AR model, as indicated at step 206. For example, the AR model coefficients Ω, K, G, and P can be extracted from the AR model. Using these AR model coefficients, a Kalman filter is constructed to estimate the neural state of the subject in response to the delivered neurostimulation, as indicated at step 208. For example, the Kalman filter can be constructed based on the following state space equations:

$${\hat{x}}_{t + 1} = ( \hat{A} - \hat{L} \hat{C} ) {\hat{x}}_{t} + ( \hat{B} - \hat{L} \hat{D} ) {u}_{t} + \hat{L} {y}_{t} ; \qquad ( 7 )$$

$${\hat{y}}_{t} = \hat{C} {\hat{x}}_{t} + \hat{D} {u}_{t} ; \qquad ( 8 )$$

where \hat{x}, is the current neural state and \hat{x}ᵢ₊₁ is the estimated neural state. The Kalman filter coefficient matrices (Â, \hat{B}, Ĉ, \hat{D}) and the Kalman gain, \hat{L}, can be constructed from the AR model coefficients Ω, K, G, and P extracted from the AR model.

The constructed Kalman filter is then used to estimate the neural state, as indicated at step 210. In parallel, the AR model makes a prediction of the neural response and the error of this prediction is fed back to the controller to update the AR model, as indicated by process loop 212. For instance, the original neural activity signals can be compared with a single step delay of the AR model signal to estimate the error. This adaptive feedback can be used to adaptively construct and update the AR model.

Updated neurostimulation settings are then calculated using the current estimate of the neural state, as indicated at step 214. For example, the neurostimulation waveform can be generated based on Eqn. (11) below.

The updated neurostimulation settings are then stored by the controller of the neurostimulation device, as indicated at step 216. For instance, the neurostimulation settings can be stored in a memory or data storage device or medium that is part of the neurostimulation device, or can be stored in memory or data storage device or medium that is remote to the neurostimulation device. In these latter instances, the neurostimulation settings can be stored for later use, at which time the neurostimulation settings can be communicated or otherwise transmitted or transferred to the neurostimulation device. The updated neurostimulation settings may then be used by the neurostimulation device to deliver the updated neurostimulation to the neural target of the subject, as indicated at steps 112 and 102 in FIG. 1.

As indicated in FIG. 2, both the neurostimulation settings and the AR model may be updated at each time step of the ARTISTS control framework. That is, steps 212 and 214 may be performed at every time step. On the other hand, the controller may be updated using a linear quadratic regulator (LQR) less frequently. For instance, the LQR may be used to update the controller every few hundred time steps. As one example, the LQR may be used to update the controller every 100 steps, every 200 steps, every 300 steps, every 400 steps, every 500 steps, every 600 steps, every 700 steps, every 800 steps, every 900 steps, every 1000 steps, and so on. Alternatively, the LQR may update the controller for any number of time steps within a suitable range, such as 100-1000 time steps.

The LQR is also constructed using the AR model coefficients in an outer loop of the controller, as indicated at step 218. For example, given a discrete time linear system:

$$x ( k + 1 ) = A ( k ) x ( k ) + B ( k ) u ( k ) ; \qquad ( 9 )$$

the LQR can be constructed based on the following quadratic cost function of state change trajectory:

$$J = \frac{1}{2} {x}^{T} ( N ) Qx ( N ) + \frac{1}{2} {\sum }_{k = 0}^{N - 1} [ {x}_{k}^{T} Qx ( N ) + {u}_{k}^{T} {Ru}_{k} ] ; \qquad ( 10 )$$

where P(k) is the cost to destination, k indicates the number of steps from the destination, Q is the cost of error, and R is the cost of the input. By taking the partial derivative of the quadratic cost function, J, with respect to u, minimizing the derivative provides the minimum activity given the stimulus u for that time step:

$${u}^{*} = Kx ; \qquad ( 11 )$$

$$K = - {[ R + {B}^{T} {P}^{*} B ]}^{- 1} {B}^{T} {P}^{*} A . \qquad ( 12 )$$

The stimulation over time based on history can then solved recursively using the discrete time algebraic Riccati equations:

$$P = {A}^{T} PA - ( {A}^{T} PB ) {( R + {B}^{T} PB )}^{- 1} ( {B}^{T} PA ) + Q . \qquad ( 13 )$$

Starting with the arbitrary initial condition of P(0):

$$P ( 1 ) = {[ A + BF ( 1 ) ]}^{T} \times P ( 0 ) [ A + BF ( 1 ) ] + {F}^{T} ( 1 ) RF ( 1 ) + Q \qquad ( 14 )$$

$$⋮$$

$$P ( k + 1 ) = {A}^{T} P ( k ) A - {A}^{T} P ( k ) {B ( {B}^{T} P ( k ) B + R )}^{- 1} {B}^{T} P ( k ) A + Q$$

$$⋮$$

$${P}^{*} = P ( k + 1 ) = P ( k ) .$$

Thus, as an example, the updated neurostimulation settings can be determined at each time step in step 214 using Eqn. (11). At every time step, the updated neural state, x, is used to calculate new neurostimulation settings. Because implementing the LQR can be more computationally intensive, K may be updated less frequently (e.g., every few hundred time steps, as indicated above).

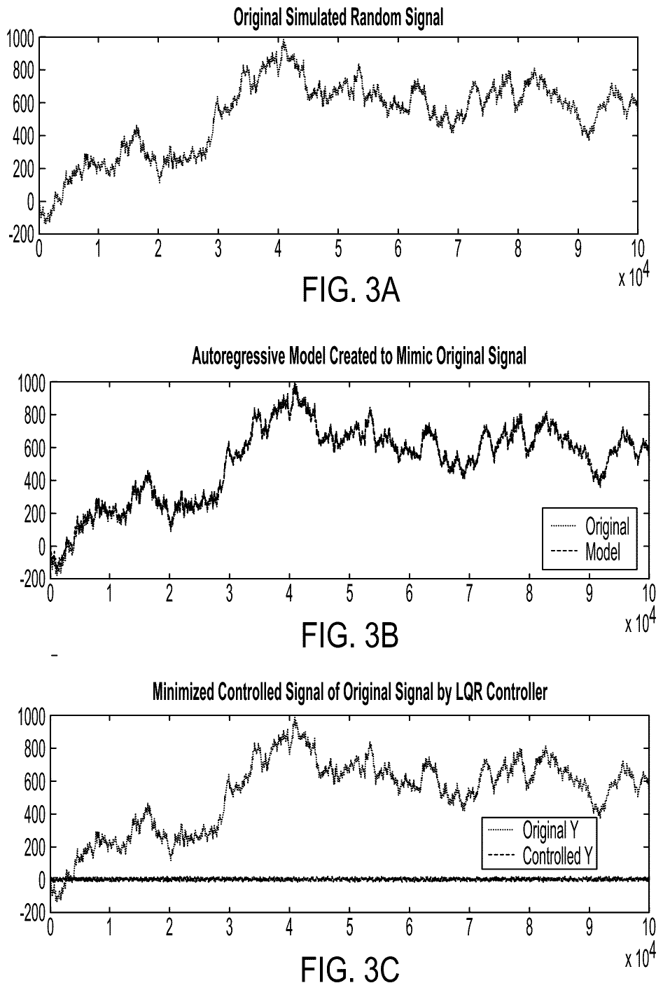

FIGS. 3A-3C illustrate an example where the ARTISTS control framework effectively minimized a signal. In FIG. 3A, a random artificial signal is simulated. In FIG. 3B, the controller builds an autoregressive model of the original signal in FIG. 3A. Lastly, the original signal is controlled and minimized by the ARTISTS control framework stimulation waveform output to produce a controlled state of the system shown in FIG. 3C.

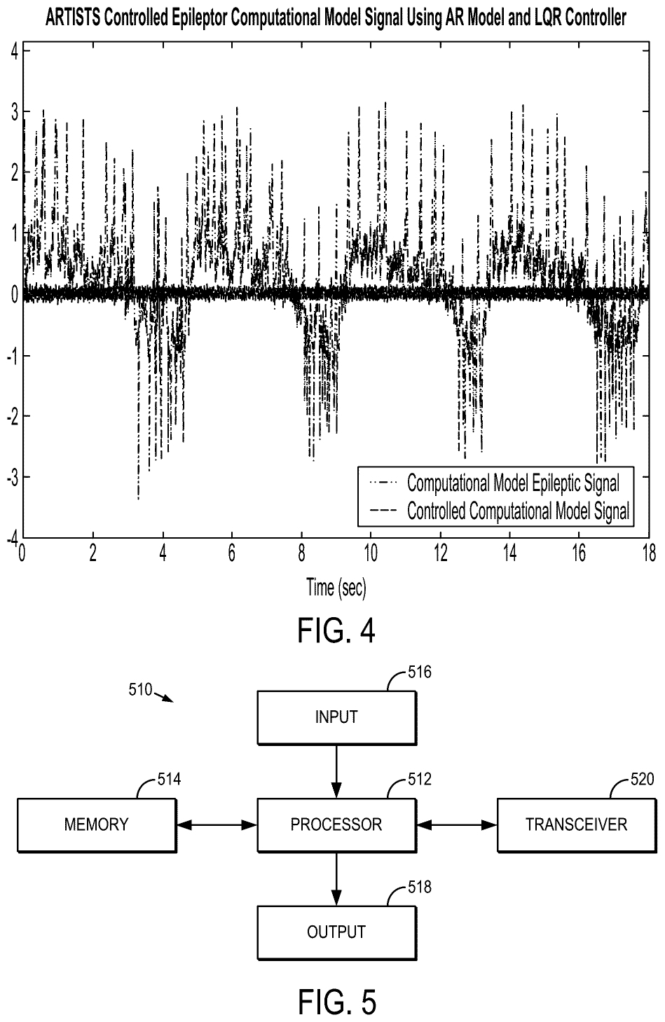

FIG. 4 illustrates an example where the ARTISTS control framework effectively minimized a signal in real time. An epileptic activity local field potential was simulated in real time using the Epileptor model. The output of the model in real time was used in the ARTISTS control framework to build an autoregressive model and solve the linear quadratic regulator equations to estimate the optimal stimulus waveform adaptively.

Referring now to FIG. 5, an example of a controller 510 that can implement the methods described in the present disclosure to control a neurostimulation device is illustrated. In general, the controller 510 includes a processor 512, a memory 514, and input 516, and an output 518. The controller 510 can be implemented as part of a neurostimulation device, or as a separate controller that is in communication with the neurostimulation device via the output 518. As one example, the controller 510 can be implemented in a neurostimulation device, such as an implantable medical device (e.g., an implanted neurostimulation system such as a deep brain stimulation (DBS) system), a standalone neurostimulation device (e.g., an external neurostimulation device, such as a transcranial magnetic stimulation (TMS) system, transcranial electrical stimulation (tES) system), and so on. In other examples, the controller 510 can be implemented in a remote computer that communicates with the neurostimulation device. In still other examples, the controller 510 can be implemented in a smartphone that is paired with the neurostimulation device, such as via Bluetooth or another wireless or wired communication.

In some embodiments, the input 516 is capable of recording neural signal data, or other physiological measurement data, from the user. As one example, the neural signal data can be electrophysiological activity data (e.g., EEG signal data, other measured neural activity data), and the input 516 can be one or more electrodes (e.g., external electrodes, implanted electrodes). The input 516 can include a wired or wireless connector for receiving neural signal data. These neural signal data can be transmitted to the controller 510 via the input 516.

The processor 512 includes at least one hardware processor to execute instructions embedded in or otherwise stored on the memory 514 to implement the methods described in the present disclosure. The memory can also store baseline signal data, measured neural signal data, one or more models, and model parameters, as well as settings to be provided to the processor 512 for generating control signals to be provided to a neurostimulation device via the output 518.

The output 518 communicates control signals to a neurostimulation device. As one example, the control signals provided to the output 518 can control one or more electrodes and/or electromagnetic coils to operate under control of the controller 510 to deliver electromagnetic stimulations (e.g., by generating electric fields, magnetic fields, or both) to the subject. Circuitry in the controller 510 can detect and processes electrophysiological activity sensed by the one or more electrodes via the input 516 to determine and/or adjust stimulation settings (e.g., stimulation waveforms) based on the methods and algorithms described in the present disclosure. The stimulation settings are provided as instructions to a pulse generator in the neurostimulation device via the output 518, which in response to the instructions provides an electrical signal to the one or more electrodes or electromagnetic coils to deliver the neurostimulations to the subject.

The controller 510 can also include a transceiver 520 and associated circuitry for communicating with a programmer or other external or internal device. As one example, the transceiver 520 can include a telemetry coil. In some embodiments, the transceiver 520 can be a part of the input 516.

In operation, the controller 510 receives neural signal data from the subject via the input 516. These neural signal data are provided to the processor 512 where they are processed. For example, the processor 512 analyzes the neural signal data using an ARTISTS control framework for determining and adjusting stimulation settings for the neurostimulation device.

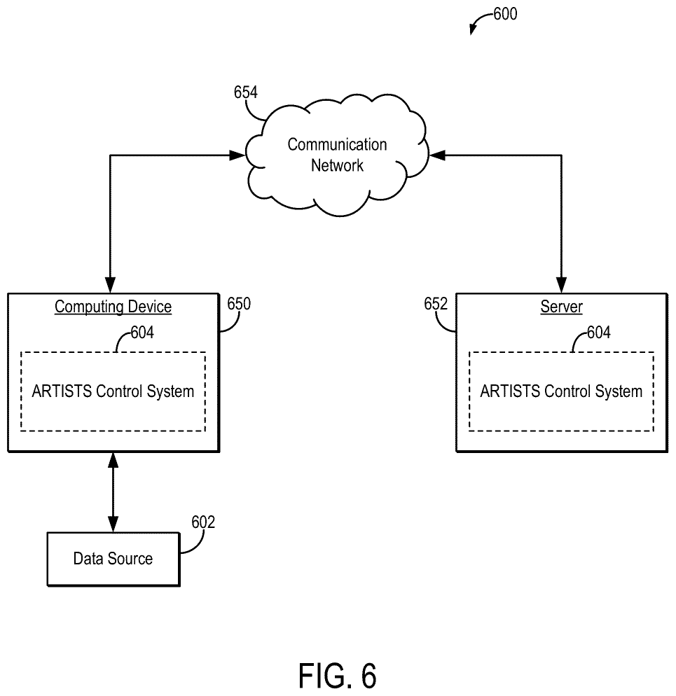

FIG. 6 shows an example of a system 600 for determining or otherwise generating neurostimulation settings in accordance with some embodiments described in the present disclosure. As shown in FIG. 6, a computing device 650 can receive one or more types of data (e.g., neural activity signal data, AR model coefficients, neural state data) from data source 602. In some embodiments, computing device 650 can execute at least a portion of an ARTISTS control system 604 to generate updated neurostimulation settings from data received from the data source 602.

Additionally or alternatively, in some embodiments, the computing device 650 can communicate information about data received from the data source 602 to a server 652 over a communication network 654, which can execute at least a portion of the ARTISTS control system 604. In such embodiments, the server 652 can return information to the computing device 650 (and/or any other suitable computing device) indicative of an output of the ARTISTS control system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. In some embodiments, the computer device 650 may be the processor 512 of the neurostimulation device controller 510.

In some embodiments, data source 602 can be any suitable source of data (e.g., neural activity signal data, processed neural activity signal data), such as recording electrodes, another computing device (e.g., a server storing neural activity signal data, processed neural activity signal data), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

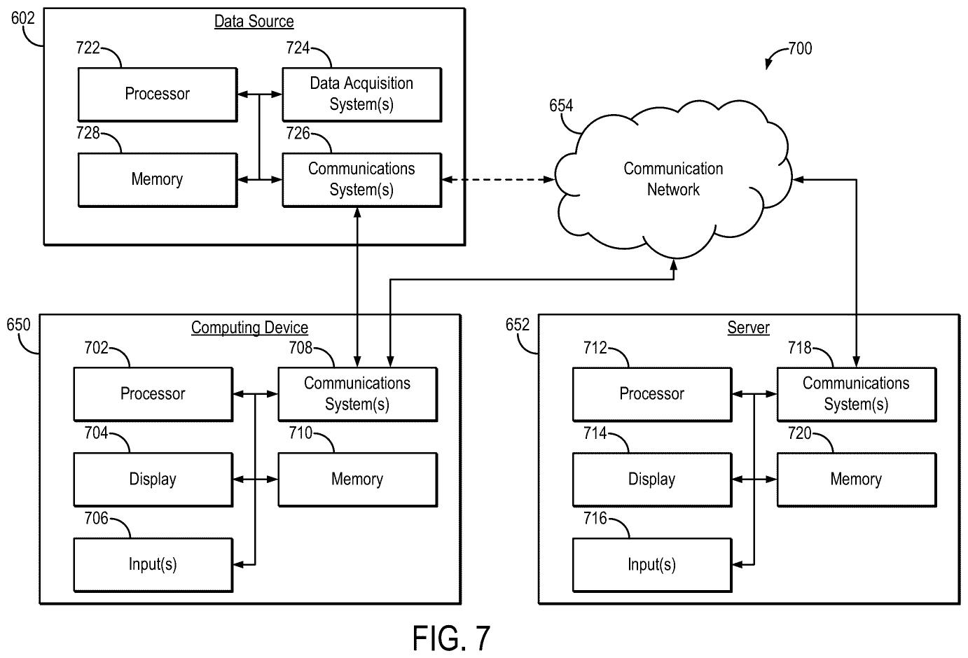

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (CPU), a graphics processing unit (GPU), and so on. In some embodiments, display 704 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the method of FIG. 1, the method of FIG. 2).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the method of FIG. e.g., the method of FIG. 1, the method of FIG. 2).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data (e.g., neural activity signal data) and can include one or more recording electrodes of a neurostimulation device, and/or one or more separate recording electrodes. Additionally or alternatively, in some embodiments, the one or more data acquisition systems 724 can include any suitable hardware, firmware, and/or software for coupling to and/or controlling operations of one or more recording electrodes. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for determining stimulation settings for a neurostimulation device, the method comprising:
(a) measuring neural activity in a subject using a recording electrode, the neural activity being in response to a neurostimulation delivered to a neural target in the subject by a neurostimulation device;
(b) constructing an autoregressive model based on the measured neural activity;
(c) estimating a neural state of the subject based on the autoregressive model, wherein the neural state comprises a real-time state of the subject's nervous system;
(d) determining stimulation settings based on the estimated neural state; and
(e) delivering neurostimulation to the neural target in the subject with the neurostimulation device using the stimulation settings.

2. The method of claim 1, wherein the autoregressive model is constructed based on the measured neural activity to minimize prediction error of a response to the neurostimulation delivered to the neural target.

3. The method of claim 1, wherein the neural state is estimated using a Kalman filter constructed based on the autoregressive model.

4. The method of claim 3, further comprising extracting model coefficients from the autoregressive model and constructing the Kalman filter using the model coefficients.

5. The method of claim 4, wherein the Kalman filter is constructed using the model coefficients to generate Kalman filter coefficient matrices and Kalman gain for the Kalman filter.

6. The method of claim 1, wherein the stimulation settings are generated using a linear quadratic regulator.

7. The method of claim 6, wherein the linear quadratic regulator is constructed using the estimated neural state to define a cost function of the linear quadratic regulator.

8. The method of claim 7, wherein the linear quadratic regulator is further constructed based on model coefficients extracted from the autoregressive model.

9. The method of claim 1, wherein the neural activity is measured using one or more recording electrodes.

10. The method of claim 9, wherein the one or more recording electrodes are part of the neurostimulation device.

11. The method of claim 1, wherein the stimulation settings comprise a neurostimulation waveform.

12. A controller for controlling a neurostimulation device, comprising:
an input that receives neural activity signals measured from a subject;
a processor to:
receive the neural activity signals from the input;
construct an autoregressive model based on the neural activity signals;
extract coefficients from the autoregressive model;
estimate a neural state based on the extracted autoregressive model coefficients, wherein the neural state comprises a real-time state of the subject's nervous system;
determine stimulation settings based on the estimated neural state; and
an output that receives the stimulation settings from the processor and communicates the stimulation settings to a neurostimulation device.

13. The controller of claim 12, wherein the autoregressive model is constructed by the processor to minimize prediction error of a response to neurostimulation delivered to a neural target in a subject.

14. The controller of claim 12, wherein the neural state is estimated using a Kalman filter constructed based on the extracted autoregressive model coefficients.

15. The controller of claim 14, wherein the processor constructs the Kalman filter by using the model coefficients to generate Kalman filter coefficient matrices and Kalman gain for the Kalman filter.

16. The controller of claim 12, wherein the processor generates the stimulation settings using a linear quadratic regulator.

17. The controller of claim 16, wherein the linear quadratic regulator is constructed by the processor using the estimated neural state to define a cost function of the linear quadratic regulator.

18. The controller of claim 17, wherein the linear quadratic regulator is further constructed by the processor based on the extracted autoregressive model coefficients.

19. The controller of claim 12, wherein the neural activity signals are received by the input from one or more recording electrodes.

20. The controller of claim 12, wherein the stimulation settings comprise a neurostimulation waveform.
